(19) United States
(12) Patent Application Publication
(10) Pub. No.: US 2006/0106074 A1
(43) Pub. Date: May 18, 2006

(54) ER-B-SELECTIVE LIGANDS

(76) Inventor: Peter Bernstein, Wilmington, DE (US)

Correspondence Address: WHITE & CASE LLP;PATENT DEPARTMENT, 1155 AVENUE OF THE AMERICAS, NEW YORK, NY, 10036, US

(21) Appl. No.: 10/497,719

(22) PCT Filed: Nov. 27, 2002
(86) PCT No.: PCT/SE02/02187

§ 371 Date: Oct. 15, 2004

(30) Foreign Application Priority Data
Nov. 28, 2001 (SE) 0103973-4

(51) Int. Cl. A61K 31/428; A61K 31/423

(52) U.S. Cl. 514/367; 514/375; 548/152; 548/217

(57) ABSTRACT

The invention relates to novel compounds having the general formula: (1) wherein X is O or S which are useful as selective ER-β ligands in the treatment or prophylaxis of Alzheimer's disease, anxiety disorders, depressive disorders, osteoporosis, cardiovascular disease, rheumatoid arthritis or prostate cancer.

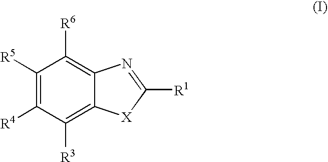

### TECHNICAL FIELD

The present invention is directed to a series of ligands, and more particularly to estrogen receptor-β ligands which have better selectivity than estrogen for the estrogen receptor-β over the estrogen receptor-β, as well as to methods for their production and use in the treatment of diseases related to the estrogen receptor-s, specifically, Alzheimer's disease, anxiety disorders, depressive disorders, osteoporosis, cardiovascular disease, rheumatoid arthritis, or prostate cancer.

### BACKGROUND

Estrogen-replacement therapy (“ERT”) reduces the incidence of Alzheimer's disease and improves cognitive function in Alzheimer's disease patients (Nikolov et al. Drugs of Today, 34(11), 927-933 (1998)). ERT also exhibits beneficial effects in osteoporosis and cardiovascular disease, and may have anxiolytic and anti-depressant therapeutic properties. However, ERT shows detrimental uterine and breast side effects that limit its use.

The beneficial effects of ERT in post-menopausal human women is echoed by beneficial effects of estrogen in models relevant to cognitive function, anxiety, depression, bone loss, and cardiovascular damage in ovariectomized rats. Estrogen also produces uterine and breast hypertrophy in animal models reminiscent of its mitogenic effects on these tissues in humans.

The beneficial effects of ERT in post-menopausal human women is echoed by beneficial effects of estrogen in models relevant to cognitive function, anxiety, depression, bone loss, and cardiovascular damage in ovariectomized rats. Specifically, experimental studies have demonstrated that estrogen effects the central nervous system (“CNS”) by increasing cholinergic function, increasing neurotrophin/neurotrophin receptor expression, altering amyloid precursor protein processing, providing neuroprotection against a variety of insults, and increasing glutamatergic synaptic transmission, among other effects. The overall CNS profile of estrogen effects in pre-clinical studies is consistent with its clinical utility in improving cognitive function and delaying Alzheimer's disease progression. Estrogen also produces mitogenic effects in uterine and breast tissue indicative of its detrimental side effects on these tissues in humans.

The estrogen receptor (“ER”) in humans, rats, and mice exists as two subtypes, ER-α and ER-β, which share about a 50% identity in the ligand-binding domain (Kuiper et al. Endocrinology 139(10) 4252-4263 (1998)). The difference in the identity of the subtypes accounts for the fact that some small compounds have been shown to bind preferentially to one subtype over the other (Kuiper et al.).

In rats, ER-β is strongly expressed in brain, bone and vascular epithelium, but weakly expressed in uterus and breast, relative to ER-α. Furthermore, ER-αa knockout (ERKO-α) mice are sterile and exhibit little or no evidence of hormone responsiveness of reproductive tissues. In contrast, ER-β knockout (ERKO-β) mice are fertile, and exhibit normal development and function of breast and uterine tissue. These observations suggest that selectively targeting ER-β over ER-α could confer beneficial effects in several important human diseases, such as Alzheimer's disease, anxiety disorders, depressive disorders, osteoporosis, and cardiovascular disease without the liability of reproductive system side effects. Selective effects on ER-β-expressing tissues (CNS, bone, etc.) over uterus and breast could be achieved by agents that selectively interact with ER-β over ER-α.

It is a purpose of this invention to identify ER-β-selective ligands that are useful in treating diseases in which ERT has therapeutic benefits.

It is another purpose of this invention to identify ER-β-selective ligands that mimic the beneficial effects of ERT on brain, bone and cardiovascular function.

It is another purpose of this invention to identify ER-β-selective ligands that increase cognitive function and delay Alzheimer's disease progression.

### SUMMARY OF THE INVENTION

This present invention is directed to compounds having the generic structure: 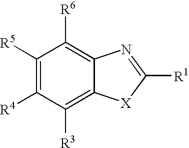
These compounds are ER-β-selective ligands, which mimic ERT, but lack undesirable side effects of ERT and are useful in the treatment or prophylaxis of Alzheimer's disease, anxiety disorders, depressive disorders, osteoporosis, cardiovascular disease, rheumatoid arthritis or prostate cancer.

### DETAILED DESCRIPTION OF THE INVENTION

The compounds of the instant invention are ER-β-selective ligands of the structure: 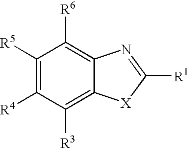
wherein:

X is O or S;

R¹ is C₁₋₈alkyl, phenyl, benzyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl, phenyl, benzyl or heterocycle is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl;

R³ is —R^{a}, —OR^{a}, —SR^{a}, —NR^{a}R^{a}, —CO₂R^{a}, —OC(═O)R^{a}, —C(═O)NR^{a}R^{a}, —NR^{a}C(O)R^{a}—NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R³ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R⁴ is H or —NR^{a}R^{b};

R⁵ is H or —NR^{a}R^{b}; wherein R⁴ and R⁵ are not both H;

R⁶ is —R^{a}, —OR^{a}, —SR^{a}, —NR^{a}R^{a}, —CO₂R^{a}, —OC(═O)R^{a}, —C(═O)NR^{a}R^{a}, NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;

R^{a} is H, C₁₋₆alkyl, C₁₋₃haloalkyl, phenyl or benzyl; and

R^{b} is C₁₋₈alkyl, C₁₋₈alkylC₄₋₈cycloalkyl, C₂₋₆alkenyl, C₂₋₆alkenyl-Ph, C₂₋₆alkenyl-Het, —(CH₂)ₙ-Ph or —(CH₂)ₙ-Het wherein n is 0-4 and Het is a 5- or 6-membered ring heterocycle containing 1, 2 or 3 heteroatoms each independently selected from O, N and S and additionally having 0 or 1 oxo groups and 0 or 1 fused benzo rings, wherein the C₁₋₄alkyl, phenyl or heterocycle is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, NR^{a}S(═O)₂R^{a} —C(═O)R^{a}, —S(═O)R^{a}, —S(═O)₂R^{a}, halogen, cyano, nitro and C₁₋₃haloalkyl.

In another embodiment R¹ is C₁₋₈alkyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl, phenyl, benzyl or heterocycle is substituted by 0, 1, 2 or 3 substituents selected from —R^{a}, —OR, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a} NR^{a}S(═O)₂R^{a}, —C(═O)R^{a}, —S(═O)R^{a}, —S(═O)₂R^{a}, halogen, cyano, nitro and C₁₋₃haloalkyl.

In another embodiment R³ is C₁₋₆alkyl, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R³ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

In another embodiment R⁶ is C₁₋₆alkyl, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a} S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

In another embodiment R¹ is phenyl or benzyl, wherein the phenyl or benzyl is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl. In a more specific embodiment, R¹ is 4-hydroxyphenyl substituted by 0, 1 or 2 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(—O)R^{a}, —S(═O)₂R^{a}, halogen, cyano, nitro and C₁₋₃haloalkyl.

In another embodiment X is S. In an alternative embodiment, X is O.

In another embodiment, R³ is halogen, cyano or C₁₋₆alkyl.

In another embodiment, R⁶ is halogen, cyano or C₁₋₄alkyl.

Preferably R⁵ is hydrogen.

Preferably R⁴ is —NR^{a}R^{b} wherein R^{a} is hydrogen, C₁₋₈ alkyl or benzyl and R^{b} is C₁₋₈ alkyl (for example methyl, ethyl or n-propyl), C₁₋₈ alkylC₄₋₈cycloalkyl (for example cyclohexylmethyl or cyclohexylethyl), C₂₋₆alkenyl (for example propen-2-yl), phenyl, phenylC₁₋₄alkyl (for example benzyl, phenethyl, phenylpropyl, phenylbutyl), Het, or HetC₁₋₄alkyl (for example imidazolylmethyl, pyridinylmethyl or thiophenylmethyl) wherein optional substituents are as described herein above.

More preferably R^{b} is 3,4-dichlorobenzyl, 3,4-diethoxybenzyl, phenethyl, 4-phenylbutyl, 3,5-dichlorobenzyl, 4-methyl-5-imidazolylmethyl, 4-(dimethylamino)-phenylmethyl, 3-phenylpropyl, 4-carboxyphenylmethyl, 3-pyridinylmethyl, 3-(2-methoxyphenyl)propyl, imidazol-4-ylmethyl, 3,5-bis(trifluoromethyl)benzyl, 4-bromo-2-thiophen-ylmethyl, 2-cyanophenylmethyl, 3-thiophen-ylmethyl, cyclohexylmethyl, 3-(4-chlorophenyl)propen-2-yl, 3-phenyl-trans-propen-2-yl, 3,3-dimethylcyclohexylethyl, 3-(4-methoxyphenyl)propen-2-yl or 4-pyridinylmethyl.

Particularly useful compounds have any of the above embodiments and also satisfy the equation:
(K_{iαA}/Ki_{βA})/(K_{iαE}/K_{iβE})>100, wherein

K_{iαA} is the Kᵢ value for the agonist in ER-α;

K_{iβA} is the Kᵢ value for the agonist in ER-β;

K_{iαE} is the Kᵢ value for estrogen in ER-α; and

K_{iβE} is the Kᵢ value for estrogen in ER-β.

Another aspect of the invention is the use of any of the above compound embodiments for the manufacture of a medicament for the treatment or prophylaxis of Alzheimer's disease, anxiety disorders, depressive disorders, osteoporosis, cardiovascular disease, rheumatoid arthritis or prostate cancer.

Another aspect of the invention is the use of any of the above compound embodiments in the treatment or prophylaxis of Alzheimer's disease, anxiety disorders, depressive disorders (including post-partum and post-menopausal depression), osteoporosis, cardiovascular disease, rheumatoid arthritis or prostate cancer.

C_{Y-Z}alkyl, unless otherwise specified, means an alkyl chain containing a minimum Y total carbon atoms and a maximum Z total carbon atoms. These alkyl chains may be branched or unbranched, cyclic, acyclic or a combination of cyclic and acyclic. For example, the following substituents would be included in the general description “C₄₋₇alkyl”:

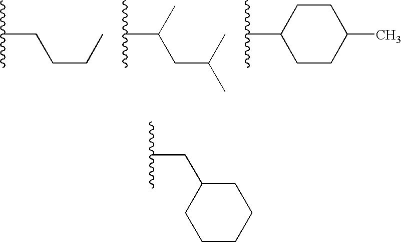

The term “oxo” means a double bonded oxygen (═O).

The compounds of the invention may contain heterocyclic substituents that are 5- or 6-membered ring heterocycles containing 1, 2 or 3 heteroatoms each independently selected from O, N and S and additionally having 0 or 1 oxo groups and 0 or 1 fused benzo rings. A nonexclusive list containing specific examples of such heterocycles are as follows: 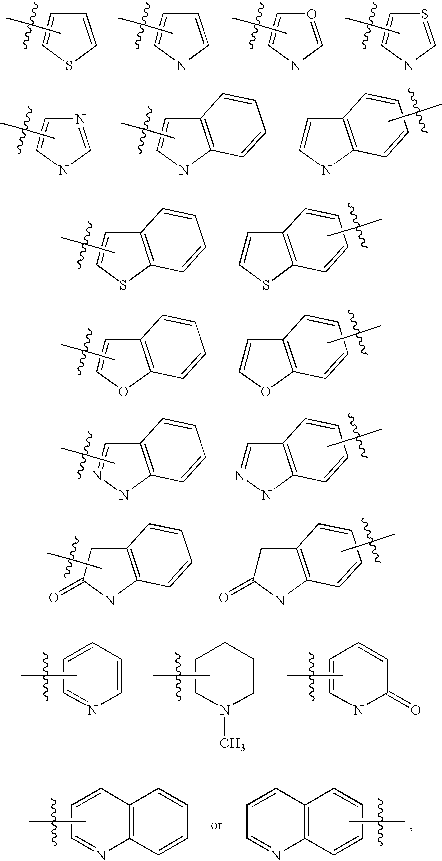
wherein the crossed bond represents that the heterocycle may be attached at any available position on either the heterocycle or the benzo ring.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bisulfate, butyrate, camphorate, camphorsulfonate, citrate, cyclohexyl sulfamate, ethanesulfonate, fumarate, glutamate, glycolate, hemisulfate, 2-hydroxyethyl-sulfonate, heptanoate, hexanoate, hydrochloride, hydrobromide, hydroiodide, hydroxymaleate, lactate, malate, maleate, methanesulfonate, 2-naphthalenesulfonate, nitrate, oxalate, pamoate, persulfate, phenylacetate, phosphate, picrate, pivalate, propionate, quinate, salicylate, stearate, succinate, sulfamate, sulfanilate, sulfate, tartrate, tosylate (p-toluenesulfonate), and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuao or by freeze drying or by exchanging the anions of an existing salt for another anion on a suitable ion-exchange resin.

Estrogen Receptor Binding Measurements

Abbreviated Procedure for Fluorescence Polarization Estrogen Receptor (ERFP) Binding Assay

A homogeneous mix-and-measure estrogen receptor (ER) binding assay which utilizes fluorescence polarization (FP) technology is used to identify compounds with affinity for the estrogen receptor. Purchased from PanVera (Madison, Wis.), assay reagents include purified human recombinant ERα, human recombinant ERβ, ES2 screening buffer (100 mM potassium phosphate, pH 7.4, 100 μg/mL bovine gamma globulin), and Fluormone™ ES2. Fluormone™ ES2, whose formulation is proprietary to PanVera, is a fluorescein-tagged, estrogen-like molecule which exhibits approximately equal affinity for ERα and ERβ.

For competition binding experiments, dilutions of test compounds are prepared at 2× the final assay concentration in 0.2% DMSO in ES2 Screening buffer on TECAN Genosys, and 25 μL compound/well is dispensed into black Costar 12 volume 96-well plates. Dependent upon a lot specific K_{d} determination, 1040 nM ERα or 10-40 nM ERβ and 1 nM Fluormone ES2 are then added to these plates in a final assay volume of 50 μL/well. Plates are gently shaken for at least 5 minutes to mix and incubated for at least 1 hr 45 minutes to achieve equilibrium. (Reaction mixtures are stable for up to 5 hours). After centrifugation to remove air bubbles, plates are read on an LJL Analyst or Acquest equipped with Criterion software at the following settings: Fluorescence Polarization Mode; Static Polarizer on Excitation Side; Dynamic Polarizer on Emission Side; Excitation λ=485+/−10 nm; Emission λ=520+/−12.5 nm.

Polarized fluorescence intensity values are collected and subsequently converted electronically to millipolarization (mp) values. Following data reduction and normalization with Excel and/or Prism software, % Ctrl values at the various test concentrations are used to obtain IC₅₀ values via non-linear regression analysis of a four-parameter logistic equation.

Because ligand depletion is a consideration in this assay (˜40-60% input ES2 is bound in the assay), IC₅₀ values are converted to Kᵢ values through application of the Kenakin formula, as outlined in the reference below, rather than via the more routinely-used Cheng-Prusoff formula.
Reference: Bolger et al., Rapid Screening of Environmental Chemicals for Estrogen Receptor Binding Capacity, Environmental Health Pespectives:106 (1998), 1-7.
   Cell-Based Assay for ER Transcriptional Activity:

ERs are ligand-dependent transcription factors that bind the promoter regions of genes at a consensus DNA sequence called the estrogen responsive element (ERE). The ER agonist or antagonist activity of a drug was determined by measuring the amount of reporter enzyme activity expressed from a plasmid under the control of an estrogen-responsive element when cells transiently transfected with ER and the reporter plasmid were exposed to drug. These experiments were conducted according to the following methods.

Plasmids:

Estrogen Receptors alpha (αER, Gen Bank accession #M12674), and beta (βER, Gen Bank # X99101 were cloned into the expression vector pSG 5 (Stratagene) and pcDNA3.1. A trimer of the vitellogenin-gene estrogen response element (vitERE) was synthesized as an oligonucleotide and attached to a beta-globin basal promoter in a construct named pERE3gal. This response element and promoter were removed from pERE3gal by digestion with the endonucleases SpeI (filled with Klenow fragment) and HindIII. This blunt/Hind III fragment was cloned into the β-galactosidase (β-gal) enhancer reporter plasmid (pBGALenh, Stratagene). αER and βER plasmids were purified using a the Endo Free Maxi Kit (Qiagen), and the DNA concentration and purity (A260/280 ratio) were determined spectrophotometrically (Pharmacia). Only DNA with A260/280 ratio of 1.8 and a concentration of >1 ug/uL was used for transfections.

Vitellogenin Response Element Sequence:

CTAGTCTCGAGAGGTCACTGTGACCT [symbol] AGGTCACTGTGACCTAGA
TCTAGGTCACTGTGACCTAC
=SpeI overhang
=XhoI site
=AfIII overhang
=ERE consensus
=

[symbol]

Cells:

All Transfections are performed in 293 cells (Human Embryonic Kidney cells ATCC # CRL-1573). Cells are grown in DMEM supplemented with 10% FBS, glutamine, sodium pyruvate and penicilin/streptomycin. Cells are grown to 80% confluency and split 1:10 or 1:20.

Transfection:

1. 293T cells are split the night before onto collagen 1150 nmn plates (Biocoat Becton Dickinson #354551) at 5 million cells per plate in phenol red-free DMEM (Mediatech 17-205-CV) 10% FBS charcoal stripped (biocell #6201-31) with supplements.
2. The next day the media is changed, 1 hour prior to transfection, to fresh phenol red-free DMEM 10% FBS (charcoal stripped) and supplements.

3. Transfections are performed using the Profection Kit from Promega #E1200, this kit is based on calcium phosphate mediated transfection. Reagents are added in sterile polystyrene tubes in the following order:

Solution A

| | |
|---|---|
| 20 | ug ER alpha or beta (in pcDNA3.1) |
| 50 | ug Reporter (pERE3 betaGal) |
| 1.5 | ML Sterile Water |
| 186 | uL CaCl2 |
| *Mix gently | |

Solution B

| | |
|---|---|
| 1.5 | ml 2XHBSS |

4. Using a vortex set on low add solution A to solution B dropwise. The resulting solution should become milky in color. It is important to get thorough mixing at this point. Let solution stand 30 min. Vortex before adding to cells.
5. Add the mixture to 150 mm plates dropwise. Mix well by rocking plates back and forth and side to side gently. View cells under 20× magnification, a very fine precipitate should be seen floating on and above cells after an hour. If you do not observe this the transfection will not work well. Incubate 18-20 hours.
   Receptor Stimulation:
1. The day after transfection cells are washed 2× with PBS Ca Mg free containing 1 mM EGTA pH=7.6. Cells are trypsinized for 5 min with 4 ml of trypsin (0.25%)-EDTA. Trypsin is neutralized with 6 ml DMEM (no phenol red)+10% charcoal stripped FBS. Cells are pelleted at 1000×g for 5 min. Cell pellet is resuspended in 10 ml DMEM (no phenol red)+2% charcoal stripped FBS supplemented with glutamine and Penn/Strep and the cells are counted. Additional medium is added to dilute the cell density to 500,000 cells/ml.
2. Cells are plated into 96 well dish (Biocoat BD #354407) at 50 ul of cells per well (=25,000 cells/well), using a multichannel pipettor. Plates are incubated for approx. 2-4 hours to allow cells to attach.
3. Compounds are prepared at concentration of 4 mM in 100% DMSO, then diluted into medium with supplements but no serum. The first 2 dilutions are done in medium with no DMSO, then the remaining dilutions are in medium plus 0.5% DMSO to keep the vehicle constant. Max controls are 10 nM beta-estradiol and background controls are 0.5% DMSO. Compounds are normally tested in the range of 10 uM to 1 nM and are prepared at twice the concentration to be tested. The compounds are added to the cell plates, 50 ul per well. All compounds are tested with an n=4 wells for single poke and n=2 for 9-pt curves.
4. Cells are incubated overnight at 37° C. with the compounds.
   Reporter Assay: I. After 18-24 hr of stimulation, 100 ul of 7% CPRG cocktail is added to each well, the plate is incubated at 37 C for approximately 30 minutes to 2 hours or until the OD reaches between 1.0 and 2.0. The CPRG (Roche 0884308) will turn bright red as Beta Gal cleaves it.
2. The plates are read on a spectrophotometric plate reader (Spectramax, Molecular Devices) at 570 nm and raw absorbances are obtained.
   Data is compiled and interpreted with Excel using XLFit or GraphPad Prism to fit concentration-response curves. The EC50 is defined as the concentration at which 50% of the fitted maximum for a compound has been reached.

### 10×Z Buffer

| | |
|---|---|
| Sodium Phosphate (dibasic) 1.7 g | 600 mM |
| Sodium Phosphate (monobasic) 0.96 g | 400 mM |
| Potassium Chloride 149 mg | 100 mM |
| Magnesium Sulfate 0.2 mL of 1 molar stock | 100 mM |
| BME 0.78 mL | 500 mM |
| Bring Final Volume to 20 mL with De-Ionized Water | |

### 7% CPRG Cocktail

For 50 mLs:

add 3.5 mL of 50 ml of CPRG

add 3.5 mL of 10×Z Buffer

add 1 mL of 10% SDS

bring to 50 mL with DI water

Typical Results:

Absorbance values illustrating typical concentration-response curves obtained for the ER agonist 17-β-estradiol (E) and the ER antagonist IC1182,780 (A) are plotted below for cells transfected with either αER or βER.

[image]

[image]

Administration and Use

Compounds of the present invention are shown to have high selectivity for ER-β over ER-α, and may possess agonist activity on ER-β without undesired uterine effects. Thus, these compounds, and compositions containing them, may be used as therapeutic agents in the treatment of various CNS diseases related to ER-β, such as, for example, Alzheimer's disease.

The present invention also provides compositions comprising an effective amount of compounds of the present invention, including the nontoxic addition salts, amides and esters thereof, which may, serve to provide the above-recited therapeutic benefits. Such compositions may also be provided together with physiologically-tolerable liquid, gel or solid diluents, adjuvants and excipients. The compounds of the present invention may also be combined with other compounds known to be used as therapeutic agents for the above or other indications.

These compounds and compositions may be administered by qualified health care professionals to humans in a manner similar to other therapeutic agents and, additionally, to other mammals for veterinary use, such as with domestic animals. Typically, such compositions are prepared as injectables, either as liquid solutions or suspensions; solid forms suitable for solution in, or suspension in, liquid prior to injection may also be prepared. The preparation may also be emulsified. The active ingredient is often mixed with diluents or excipients which are physiologically tolerable and compatible with the active ingredient. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

The compositions are conventionally administered parenterally, by injection, for example, either subcutaneously or intravenously. Additional formulations which are suitable for other modes of administration include suppositories, intranasal aerosols, and, in some cases, oral formulations. For suppositories, traditional binders and excipients may include, for example, polyalkylene glycols or triglycerides; such suppositories may be formed from mixtures containing the active ingredient. Oral formulations include such normally employed excipients as, for example, pharmaceutical grades of mannitol, lactose, starch, magnesium stearate, sodium saccharin, cellulose, magnesium carbonate, and the like. These compositions take the form of solutions, suspensions, tablets, pills, capsules, sustained-release formulations, or powders.

In addition to the compounds of the present invention that display ER-β activity, compounds of the present invention can also be employed as intermediates in the synthesis of such useful compounds.

Synthesis

Compounds within the scope of the present invention may be synthesized chemically by means well known in the art. The following Examples are meant to show general synthetic schemes, which may be used to produce many different variations by employing various commercially available starting materials. These Examples are meant only as guides on how to make some compounds within the scope of the invention, and should not be interpreted as limiting the scope of the invention.

### REFERENCE EXAMPLE 1

### 6-Bromo-2-(4-methoxy-phenyl)-benzothiazole

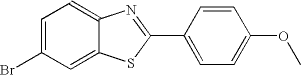

### a. N-(4-Bromo-phenyl)-4-methoxy-benzamide

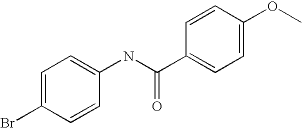 To a solution containing 4-bromo-aniline (1.0 g, 7 mmol) in pyridine (7 mL) was added p-anisoyl chloride (0.77 mL, 7.1 mmol) dropwise under nitrogen. The reaction was stirred at room temp for 30 min. Reaction was poured cautiously into saturated NaHCO₃ and extracted with ethyl acetate. Ethyl acetate extracts were washed with: 1) saturated NaHCO₃, 2) saturated brine and concentrated in vacuo. This solid was washed with a solution containing: ether/hexane (1:5, 10 mL), dried under vacuum, yielding the title compound (1.97 g, 92%) as a white solid. Mass spec: MH⁺=306.

### b. N-(4-Bromo-phenyl)-4-methoxy-thiobenzamide

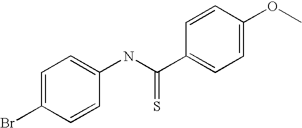 N-(4-Bromo-phenyl)₄-methoxy-benzamide (1.95 g, 6.37 mmol) and Lawesson's reagent (1.55 g, 3.82 mmol) were suspended in chlorobenzene (25 mL) and heated to reflux under nitrogen for 3.0 h. Reaction was cooled, solvent removed under vacuum. Solid was dissolved in ethyl acetate and washed with: 1) hydrochloric acid (1.0M), 2) saturated brine and concentrated in vacuo. Residue was further purified by chromatography on silica yielding the title compound (1.85 g, 90%) as a yellow-orange solid. Mass spec: MH⁺=322

### c. 6-Bromo-2-(4-methoxy-phenyl)-benzothiazole

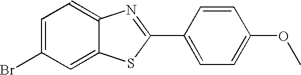 N-(4-Bromo-phenyl)-4-methoxy-thiobenzamide (483 mg, 1.5 mmol) was wetted with ethanol (4.0 mL). 30% Aqueous sodium hydroxide (10 M, 1.2 mL) was added and stirred for 5 min. Water (2.4 mL) was added to provide a final suspension of 10% aqueous sodium hydroxide. Aliquots (1 mL) of this mixture were added at 1 min intervals to a heated (85° C.) stirred solution containing potassium ferricyanide (0.98 g, 6 mmol) in water (25 mL). Reaction was kept at 85° C. for 30 min, and then cooled to room temp. Cold water (200 mL) was added. Mixture was allowed to sit undisturbed for 30 min. Precipitate was collected by filtration, washed with water, and dried under vacuum. Solid was dried under vacuum at 37° C. yielding the title compound (0.45, 93%) as a pale yellow solid. Mass spec: MH⁺=320

### REFERENCE EXAMPLE 2

### 6-Amino-2-(4-hydroxyphenyl)-benzothiazole

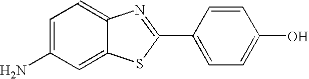 Benzyl-[2-(4-methoxy-phenyl)-benzothiazol-6-yl]-amine (225 mg, 0.65 mmol)) and pyridine hydrochloride (2.25 g, 19.5 mmol) were heated to 200° C. under nitrogen for 90 min, and then cooled to room temp. Reaction was poured cautiously into saturated NaHCO₃ and extracted with ethyl acetate. Ethyl acetate extracts were washed with: 1) saturated NaHCO₃, 2) saturated brine and concentrated in vacuo. Residue was washed with hexane, and dried under vacuum yielding the title compound (157 mg, 100%) as a yellow-orange solid. Mass spec: MH⁺=242. The starting benzyl-[2-(4-methoxy-phenyl)-benzothiazol-6-yl]-amine was prepared as follows:

### Benzyl-[2-(4-methoxy-phenyl)-benzothiazol-6-yl]-amine

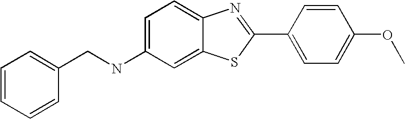 6-Bromo-2-(4-methoxy-phenyl)-benzothiazole (2.0 g, 6.25 mmol), tris (dibenzylideneacetone) dipalladium (0) (143 mg, 0.156 mmol), 2,2′-bis (diphenylphosphino)-1,1-binaphthyl (778 mg, 1.25 mmol), and sodium t-butoxide (1.8 g, 18.75 mmol) were suspended in dry THF (85 mL) under nitrogen. Benzylamine (0.804 g, 7.5 mmol) was added and reaction was heated to reflux for 18 h. Reaction was cooled, poured into saturated NaHCO₃ and extracted with ethyl acetate. Ethyl acetate extracts were washed with: 1) saturated NaHCO₃, 2) saturated brine and concentrated in vacuo. Residue was further purified by chromatography on silica yielding the title compound (1.60 g, 74%) as a yellow solid. Mass spec: MH⁺=347

### REFERENCE EXAMPLE 3

### Benzhydrylidene-[2-(4-methoxyphenyl)benzothiazol-6-yl)amine

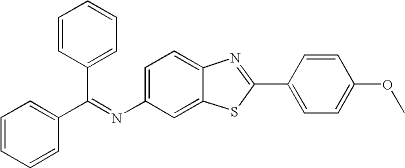 6-Bromo-2-(4-methoxy-phenyl)-benzothiazole (1.0 g, 3.125 mmol), tris (dibenzylideneacetone) dipalladium (0) (14.65 mg, 0.016 mmol), 2,2′-bis (diphenylphosphino)-1,1′-binaphthyl (38.9 mg, 0.0625 mmol), benzophenone imine (0.68 g, 3.75 mmol) and sodium t-butoxide (0.6 g, 6.25 mmol) were suspended in dry toluene (12 mL) under nitrogen and reaction was heated to 80° C. for 72 h. Reaction was cooled, poured into saturated NaHCO₃ and extracted with ethyl acetate. Ethyl acetate extracts were washed with: 1) saturated NaHCO₃, 2) saturated brine and concentrated in vacuo. Residue was further purified by chromatography on silica yielding the title compound (1.25 g, 95%) as an orange solid. Mass spec: MH⁺=421

### REFERENCE EXAMPLE 4

### 2-(4-Methoxyphenyl)benzothiazol-6-ylamine

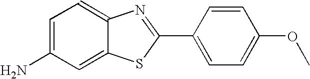 Benzhydrylidene-[2-(4-methoxy-phenyl)-benzothiazol-6-yl)-amine (0.82 g, 1.95 mmol) was dissolved in THF (35 mL) containing hydrochloric acid (2N, 5 mL) and stirred at room temp for 30 min. Reaction was poured into saturated NaHCO₃ and extracted with ethyl acetate. Ethyl acetate extracts were washed with: 1) saturated NaHCO₃, 2) saturated brine and concentrated in vacuo. Residue was washed with a solution containing hexane/ethyl acetate (5:1, 50 mL), and dried under vacuum yielding the title compound (0.50 g, 100%) as a tan solid. Mass spec: MH⁺=256

This compound can be converted to 2-(4-hydroxyphenyl)benzothiazol-6-ylamine by boron tribromide reduction in conventional manner.

The title compounds of Reference Examples 2 and 4 are useful chemical intermediates but also have pharmaceutical activity (as described in relation to the compounds of the formula (I)) in their own right.

General Reductive Amination Procedure: (See Reaction 1)

2-(4-Hydroxy-phenyl)-benzothiazol-6-ylamine (100 mg, 0.413 mmol) and aldehyde (0.7 mmol, see table 1) were dissolved in a solution containing THF (10 mL) and methanol (2 mL) under nitrogen. Crushed molecular sieves (3 Angstrom, 0.35 g) were added and stirred at room temp for 15 min. Sodium cyanoborohydride (40 mg, 0.64 mmol) was added and reaction stirred for 1 h. Glacial acetic acid (3 drops) was added and reaction was stirred at room temp for 24 h. Reactions were monitored by tlc. Additional glacial acetic acid and sodium borohydride were added if reaction was not complete in 24 h. Most reactions were completed in 72 h. When reaction was completed, add methanol (3 ml) and stir for 1 h. Reactions were worked up using one of the following methods:

Method A: Reaction was poured into saturated NaHCO₃ and extracted eith ethyl acetate. Ethyl acetate extracts were washed with: 1) saturated NaHCO₃, 2) saturated brine and concentrated in vacuo. This material was further purified by chromatography on silica yielding the title compound.

Method B: Triethylamine (0.5 mL, 3.59 mmol) was added and reaction stirred for 30 min. Solvent was removed under vacuum and residue was further purified by chromatography on silica yielding the title compound.

Method C: Reaction was poured into saturated brine and extracted with ethyl acetate. Ethyl acetate extracts were washed with: 1) hydrochloric acid (1.0M), 2) saturated brine and concentrated in vacuo. Residue was washed with methylene chloride, and dried under vacuum yielding the title compound

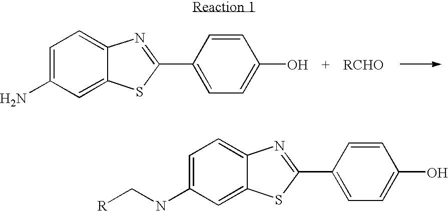

TABLE 1

| | | WORK- | | |
|---|---|---|---|---|
| | | UP | PRODUCT | YIELD |
| EXAMPLE | R | METHOD | M.W. | (%) |
| 1 | 3,4-dichlorophenyl | B. | 401 | 37 |
| 2 | 3,4-diethoxyphenyl | B. | 420 | 54 |
| 3 | benzyl | A. | 346 | 78 |
| 4 | 4-phenylpropyl | A. | 374 | 79 |
| 5 | 3,5-dichlorophenyl | B. | 401 | 100 |
| 6 | 4-methyl-5-imidazolyl | B. | 336 | 60 |
| 7 | 4-(dimethylamino)- | B. | 375 | 43 |
| | phenyl | | | |
| 8 | 2-phenylethyl | B. | 360 | 36 |
| 9 | 4-carboxyphenyl | C. | 376 | 68 |
| 10 | 3-pyridinyl | B. | 333 | 34 |
| 11 | 2-(2-methoxyphenyl) | B. | 388 | 30 |
| | ethyl | | | |
| 12 | imidazol-4-yl | B. | 322 | 11 |
| 13 | 3,5-bis(trifluoro- | B. | 468 | 36 |
| | methyl)-phenyl | | | |
| 14 | 4-bromo-2- | B. | 417 | 40 |
| | thiophene-yl | | | |
| 15 | 2-cyanophenyl | B. | 357 | 36 |
| 16 | 3-thiophene-yl | B. | 338 | 44 |
| 17 | cyclohexyl | B. | 338 | 64 |
| 18 | 2-(4-chlorophenyl)- | B. | 392 | 37 |
| | ethenyl | | | |
| 19 | 2-phenyl-trans- | B. | 358 | 51 |
| | ethenyl | | | |
| 20 | 3,3-dimethyl- | B. | 380 | 40 |
| | cyclohexylmethyl | | | |
| 21 | 2-(4-methoxyphenyl)- | B. | 388 | 37 |
| | ethenyl | | | |
| 22 | 4-pyridinyl | B. | 333 | 39 |

The molecular weights were determined via LC-MS. This was achieved using a Waters MicroMass spectrometer in positive ion APCI mode, coupled with an HP-1100 HPLC [high pressure liquid chromatograph] with a diode array detector.

1. A compound of the formula: 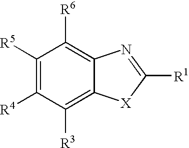
wherein:
X is O or S;
R¹ is C₁₋₈alkyl, phenyl, benzyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl, phenyl, benzyl or heterocycle is substituted by 0, 1, 2 or 3 substituents selected from —R^{a}, —OR^{a}, —SR^{a}, —NR^{a}R^{a}, CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl;
R³ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R³ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a} —NR^{a}S(═O)₂R^{a}, —C(═O)R^{a}, —S(═O)R^{a}, —S(═O)₂R^{a}, halogen, cyano and nitro;
R⁴ is H or —NR^{a}R^{b};
R⁵ is H or —NR^{a}R^{b}; wherein R⁴ and R⁵ are not both H;
R⁶ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro;
R^{a} is H, C₁₋₆alkyl, C₁₋₃haloalkyl, phenyl or benzyl; and
R^{b} is C₁₋₈alkyl, C₁₋₈alkylC₄₋₈cycloalkyl, C₂₋₆alkenyl, C₂₋₆alkenyl-Ph, C₂₋₆alkenyl-Het, —(CH₂)ₙ-Ph or —(CH₂)ₙ-Het wherein n is 0-4 and Het is a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl, phenyl or heterocycle is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or a pharmaceutically acceptable salt or hydrolyzable ester thereof.

2. The compound according to claim 1, wherein R³ is halogen, cyano or C₁₋₆alkyl.

3. The compound according to claim 1, wherein R⁶ is halogen, cyano or C₁₋₆alkyl.

4. The compound according to claim 1, wherein R¹ is C₁₋₈alkyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈alkyl, phenyl, benzyl or heterocycle is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl.

5. The compound according to claim 1, wherein X is S.

6. The compound according to claim 1, wherein R³ is C₁₋₆alkyl, OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R³ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

7. The compound according to claim 1, wherein R⁶ is C₁₋₆alkyl, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, S(═O)R^{a}, —S(═O)₂R^{a}, halogen, cyano, nitro and C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro.

8. The compound according to claim 1, wherein X is O.

9. The compound according to claim 1, wherein R¹ is phenyl or benzyl, wherein the phenyl or benzyl is substituted by 0, 1, 2 or 3 substituents selected from —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl.

10. The compound according to any one of claims 1-9, wherein the compound satisfies the equation:
(K_{iαS}/K_{iβA})/(K_{iαE}K_{iβE})>100, wherein
K_{iαA} is the Kᵢ value for the agonist in ER-α;
K_{iβA} is the Kᵢ value for the agonist in ER-β;
K_{iαE} is the Kᵢ value for estrogen in ER-α; and
K_{iβE} is the Kᵢ value for estrogen in ER-β.

11. (canceled)

12. A method for the treatment or prophylaxis of Alzheimer's disease, anxiety disorders, depressive disorders, osteoporosis, cardiovascular disease, rheumatoid arthritis or prostate cancer which comprises administering to a patient in need of such treatment or prophylaxis a therapeutically effective amount of a compound according to any one of claims 1-9.

13. A pharmaceutical composition comprising a compound according to any one of claims 1-9 and a pharmaceutically acceptable carrier.

14. A method for the treatment or prophylaxis of Alzheimer's disease, anxiety disorders, depressive disorders, osteoporosis, cardiovascular disease, rheumatoid arthritis or prostate cancer which comprises administering to a patient in need of such treatment or prophylaxis a therapeutically effective amount of a compound according to claim 10.

15. A pharmaceutical composition comprising a compound according to claim 10 and a pharmaceutically acceptable carrier.
